(19) United States
(12) Patent Application Publication
(10) Pub. No.: US 2024/0090995 A1
(43) Pub. Date: Mar. 21, 2024

(54) METHODS AND SYSTEMS FOR DETERMINING INTRAOCULAR LENS PARAMETERS FOR OPHTHALMIC SURGERY USING AN EMULATED FINITE ELEMENTS ANALYSIS MODEL

(71) Applicant: Alcon Inc., Fribourg (CH)

(72) Inventors: Robert Dimitri Angelopoulos, San Jose, CA (US); William Jacob Spenner Dolla, Plano, TX (US); Bryan Stanfill, Mansfield, TX (US)

(21) Appl. No.: 18/463,168

(22) Filed: Sep. 7, 2023

Related U.S. Application Data
(60) Provisional application No. 63/407,289, filed on Sep. 16, 2022.

(51) Int. Cl. A61F 2/16; A61B 3/00; G16H 20/00; G16H 50/70

(52) U.S. Cl. CPC A61F 2/16; A61B 3/0025; G16H 20/00; G16H 50/70

(57) ABSTRACT

Certain aspects of the present disclosure provide techniques for performing surgical ophthalmic procedures, such as cataract surgeries. An example method of determining one or more intraocular lens (IOL) parameters for an IOL to be used in a cataract surgery procedure includes generating, using a fused machine learning model, recommendations including one or more IOL parameters for the IOL to be used in the cataract surgery based, at least in part, on first predicted lens behavior for each of one or more IOLs by an emulated finite element analysis (EFEA) model and the second predicted lens behavior for each of the one or more IOLs by an IOL power calculator (IPC) machine learning model.

### INTRODUCTION

Aspects of the present disclosure relate to ophthalmic surgery, and more specifically to determining intraocular lens (IOL) parameters for an IOL to be used during cataract surgery for a patient using an emulated finite element (FEA) model. As defined herein, IOL parameters include at least one of the type, size, and power of an IOL that is to be implanted in a patient's eye during cataract surgery.

### BACKGROUND

Ophthalmic surgery generally encompasses various procedures performed on a human eye. These surgical procedures may include, among other procedures, cataract surgery. Cataract surgery is a procedure in which the crystalline or natural lens of a human eye is removed and replaced with a synthetic lens, also known as an IOL, to rectify vision problems arising from opacification of the natural lens.

Selecting the right IOL for the patient is pivotal in achieving an optimal post-operative visual outcome. IOLs come in various types, powers, and sizes and may be selected based on measurements of anatomical parameters of a patient's eye. The anatomical parameters of the human eye, such as the axial length (i.e., the distance between the anterior cornea and the retina), corneal thickness, anterior chamber depth (i.e., the distance between the anterior cornea and the anterior lens surface), and white-to-white diameter (i.e., the distance between the corneal and scleral boundary on either side of the eye), generally influence IOL parameter selections made in the planning and performing of cataract surgery on a patient. Planning and performing cataract surgery, as defined herein, includes determining the right IOL parameters for improving the patient's vision. As an example, based on the patient's measurements of anatomical parameters, a surgeon may try to determine the IOL parameters that have a high likelihood of restoring the patient's vision. The surgeon then selects an IOL, from a set of IOLs, whose parameters match the determined IOL parameters. Subsequently, the surgeon places or implants the selected IOL in the patient's lens capsule.

The measurements of anatomical parameters for a specific patient, in many cases, may be within a known distribution (e.g., between a lower bound and an upper bound where some set percentage of patients are within, such as a normal distribution of two standard deviations from a global mean in which measurements for about 95 percent of patients lie) and, therefore, planning and performing cataract surgery for such a patient may be a relatively straightforward task. However, if one or more anatomical parameters for a specific patient deviate from the known distribution or are otherwise abnormal (hereinafter “anomalous”), planning and performing cataract surgery for such a patient may be a more complicated task. Additionally, some cases may exist where the combination of anatomical parameters makes treatment of the eye a complicated task.

An IOL power calculator (IPC) may, therefore, be used to determine IOL parameters for each specific patient. The IPC may provide predictions of post-operative refractive outcomes based on a model or formula that takes the measurements of the patient's anatomical parameters as input and, for one or more different IOLs, provides the predicted post-operative refractive outcome(s) for the patient. Using the predictions from the IPC, a surgeon may try to select the IOL with the highest likelihood of restoring the patient's vision, such as the IOL with the smallest predicted post-operative refractive error.

Such IPC models or formulas have been developed for non-accommodating IOLs, but may not perform well for accommodating IOLs. Accommodating IOLs are different from standard, “static”, IOLs because they are able to change focus distances. Accommodating IOLs may be fluid-filled, allowing the lens to dynamically change shape. Accommodating IOLs have flexible “arms” called haptics, which use the movements of the eye's muscles to change focus from distance to near. This adjustment allows incoming light rays to focus properly on the retina. When the eyes gaze at a near object, the eye accommodates. During accommodation, the ciliary muscles of the eye contract, causing zonules to relax, allowing the shape of the natural lens to thicken. The thicker lens has a steeper curvature that is better able to focus incoming light rays from a near object onto the retina. With an accommodating IOL implanted in a patient's eye, when the ciliary muscles contract, this results in the bending of the flexible haptics of the accommodating IOL which causes the focusing area of the accommodating IOL to move forward. The forward position of the focusing area provides added ability to focus at near, or to “accommodate.” These complex interactions of accommodating IOLs with the components of the patient's eye may not be captured by current cataract planning and IPC(s), which may lead the surgeon to select IOL parameters that may lead to an unsuccessful surgical outcome.

Accordingly, techniques are needed for accurately determining IOL parameters for a patient based, at least in part, on measurements of anatomical parameters of the patient's eye.

### BRIEF SUMMARY

Certain embodiments provide a method for determining one or more intraocular lens (IOL) parameters for an IOL to be used in a cataract surgery procedure. The method generally includes generating, using one or more ophthalmic imaging devices, a plurality of data points associated with measurements of a plurality of anatomical parameters for an eye to be treated; generating, using a machine learning model trained to emulate a finite elements analysis (FEA) model, first predicted lens behavior based, at least in part, on the plurality of data points associated with the measurements of the plurality of anatomical parameters for the eye to be treated and one or more IOL parameters for each of one or more IOLs; generating, using an IOL power calculator machine learning model, second predicted lens behavior based, at least in part, on at least a subset of the plurality of data points associated with the measurements of the plurality of anatomical parameters for the eye to be treated and one or more IOL parameters for each of one or more IOLs; and generating, using a fused machine learning model, recommendations including one or more IOL parameters for the IOL to be used in the cataract surgery based, at least in part, on first and second predicted lens behavior.

Aspects of the present disclosure provide means for, apparatus, processors, and computer-readable mediums for performing the methods described herein.

To the accomplishment of the foregoing and related ends, the one or more aspects comprise the features hereinafter fully described and particularly pointed out in the claims. The following description and the appended drawings set forth in detail certain illustrative features of the one or more aspects. These features are indicative, however, of but a few of the various ways in which the principles of various aspects may be employed.

### BRIEF DESCRIPTION OF THE DRAWINGS

The appended figures depict certain aspects of the one or more embodiments and are therefore not to be considered limiting of the scope of this disclosure.

FIG. 1 depicts an example environment in which an emulated finite elements analysis (EFEA) model, an IPC machine learning (NIL) model, and a fused ML model are trained and deployed for use in generating recommendations, including recommended IOL parameters, for a patient's cataract surgery based at least on measurements of the patient's anatomical parameters, in accordance with certain aspects described herein

FIG. 2 is a diagram of a model eye and characteristics of the eye.

FIG. 3 is a flow diagram illustrating example operations for training of an EFEA model, in accordance with certain aspects described herein.

FIG. 4 illustrates use of an EFEA model and an IPC ML model to train a fused ML model, in accordance with certain aspects described herein.

FIG. 5 illustrates example operations that may be performed by a computing device to generate and output recommendations, including IOL parameters, for a patient's cataract surgery based on the patient's anatomical parameters, in accordance with certain aspects described herein.

FIG. 6 illustrates an example computing device in which embodiments of the present disclosure can be performed, in accordance with certain aspects described herein.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

As discussed above, cataract surgery is a surgical procedure in which a defective natural lens is replaced with an IOL. Typically, a defective natural lens is a lens that has developed a cataract, which is an opacification of the natural lens that negatively affects the patient's vision (e.g., causing a patient to see faded colors, have blurry vision or double vision, see haloing around point light sources, or other negative effects). An IOL may be selected to replace the patient's natural lens in order to restore, or at least improve, the patient's vision. The determination of a set of IOL parameters, for an IOL to be implanted in the patient's eye, are influenced by measurements of the patient's anatomical parameters. More specifically, as discussed above, based on the characteristics and/or measurements of the anatomical parameters of the patient's eye, a surgeon may try to determine a set of IOL parameters that have a high likelihood of restoring the patient's vision. For example, some IOLs may allow for optimized near or far vision, while other IOLs may be used to compensate for a patient's natural corneal astigmatism, and so on.

Accommodating IOLs are different from non-accommodating IOLs in that accommodating IOLs are able to dynamically change shape in order to change focus distances to accommodate movements of the patient's eye. In the human eye, a circular ciliary muscle surrounds the lens. Ciliary zonules connect the ciliary muscle to the lens capsule that encloses the lens. When the eyes gaze at an object at a distance, the ciliary muscle relaxes causing the ciliary zonules to become taut, which causes the lens to have a flatter curve that is better able to focus incoming light rays from distant objects onto the retina. In a young eye, accommodation is essentially instantaneous and effortless. As the eye ages, the lens becomes less flexible causing the loss of near vision that is the hallmark sign of presbyopia in people over the age of, e.g., forty. Such patients may be good candidates for using an accommodating lens. With an accommodating IOL implanted in a patient's eye, when the patient's ciliary muscles contract this results in the bending of the flexible haptics of the accommodating IOL, which causes the focusing area of the accommodating IOL to move forward providing added ability to focus.

As discussed above, the interactions of accommodating IOLs with the patient's eye may be complex and not accounted for by existing lens fitting procedures and IPCs. Accordingly, aspects presented herein provide systems and methods in which FEA is used to model interactions of the patient's eye with IOLs in order better predict IOL behavior for predicting post-operative outcomes and recommending IOL parameters for the patient. In particular, FEA may be used to train an EFEA for informing the selection of an accommodating IOL for a patient based on the patient's anatomical characteristics and/or measurements.

Finite element analysis can be used to form predictions for both static and accommodating IOLs. FEA simulates a physical phenomenon (e.g., interactions of IOLs in the human eye) using a numerical technique called the Finite Element Method (FEM). FEA may involve creating a mesh, consisting of up to millions of smaller elements (e.g., components of the eye) that together form the shape of the structure (e.g., the patient's eye). Calculations are performed for each element and the individual results are combined to provide a final result of the structure. The FEM uses partial differential equations (PDEs) and/or matrices to describe complex physical behaviors, such as structure, fluid, and thermal behaviors (e.g., behavior of an accommodating IOL in a patient's eye). These PDEs or matrices can be solved to compute relevant quantities, in order to estimate behavior of IOLs under various conditions.

The FEA can be used to model how an accommodating IOL will behave after the accommodating IOL is implanted in the patient's capsular bag to understand how different geometries of the lens capsule impact the dynamics of the accommodating IOL in order to predict a post-operative outcome for the patient for a given IOL. According to embodiments of the present disclosure, a FEA model is designed and solved for using one or more specific patients' pre-operative measurements and one or more lens designs in order to predict the behavior of the one or more lenses after implantation in the patient's eye. The FEA model can be fine-tuned by comparing predictions outputted by the FEA model to clinical data over a large design space of patient pre-operative measurements and IOL designs to predict how the IOL power of different sized fluid accommodating IOLs will change when they are implanted in different capsular bags of different patients' eyes with different geometries.

The FEA model, however, may be computationally expensive and can take, for example, up to twenty-four hours or even longer to run. In addition, the FEA model may require a specialist to generate, set up, and run the FEA model.

Accordingly, aspects of the present disclosure provide for training and use of an emulated FEA model. Use of an emulated FEA model enables a faster, more efficient, approach to leveraging FEA modeling for predicting post-operative outcomes and recommending IOL parameters for a patient. In some embodiments, the emulated FEA model is trained to match, or closely match, the output of the FEA model. The trained emulated FEA model may be less computationally expensive to run than the FEA model (e.g., the model may run in a matter of seconds) and does not require a specialist to operate. Accordingly, the emulated FEA may be portable and easily deployed in a clinical setting for a clinician or surgeon to fit a patient with an IOL for implantation.

In some embodiments, an IPC is used in addition to the emulated FEA model in order to provide predicted post-operative outcomes for a patient for a given set of IOLs with different IOL powers, types, and/or sizes. The IPC may be a standard IPC (e.g., a Bartlett IPC, a Haigis formula IPC, a Hill-RBF IPC, an SRK (Sanders-Retzlaff, Kraff) IPC, or the like). In some other embodiments, the IOL power calculator is a machine learning (ML) based IOL power calculator. As discussed in more detail herein, the ML based IPC may be trained over a large design space of patient pre-operative measurements and IOL designs to predict, for each patient, post-operative refractive errors for various accommodating IOLs based on the patient's anatomical measurements and/or characteristics.

In some embodiments, the emulated FEA model and the ML based IPC model are used to generate and train a fused model. In some embodiments, the fused model is also a ML based model. The fused model takes the output from both the emulated FEA model and the ML based IPC model as input to the fused model and then outputs recommended IOL parameters. The fused model may be trained with inputs from the emulated FEA model and the IOL power calculator to make recommendations of IOL parameters. These recommendations can be compared against clinical data to refine the fused model.

### Example Computing Environment for Ophthalmic Surgery Procedure Planning Using an EFEA Model

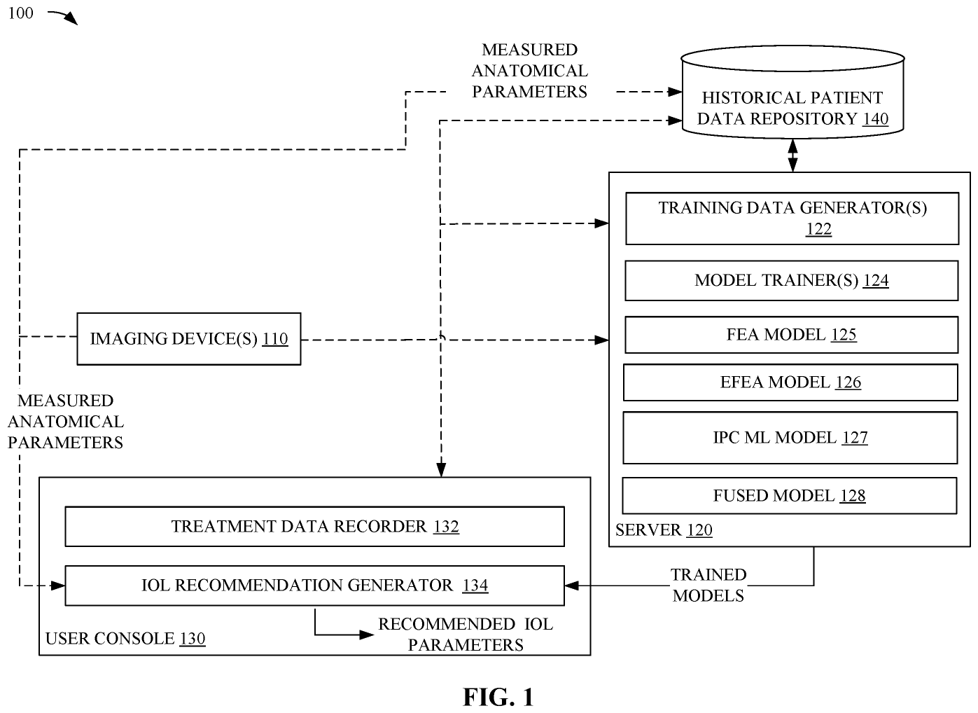

FIG. 1 illustrates an example computing environment in which models are trained and used in generating recommendations, including IOL parameters, for a patient's cataract surgery. Generally, these models may be trained using a corpus of training data including records corresponding to historical patient data and deployed for use in cataract surgery planning for a current patient, including generating IOL parameters. As defined herein, a new or current patient (hereinafter “current”) is generally a patient who is having cataract surgery to replace a defective natural lens. As discussed in further detail below, the recommended IOL parameters for the current patient may be generated by a fused model that is trained to generate recommended IOL parameters.

Historical patient data for each historical patient may include the patient's demographic information, recorded data points associated with measurements of the patient's anatomical parameters, desired outcomes, actual treatment data such as actual IOL parameters of the IOL that was implanted in the patient's eye, or other information about the historical patient's treatment, and the treatment result data (e.g., post-operative refractive error and parameters indicating the historical patient's satisfaction or dissatisfaction with the treatment (e.g., patient satisfaction score). Note that, herein, the actual treatment performed on the patient may be different than the recommended treatment.

By using these models, a large universe of historical patient data can be leveraged to generate recommended IOL parameters for the current patient. This large universe of historical patient data is, in a way, indicative of the expertise and prior experiences of other surgeons who have handled similar surgeries for similar patients. By using the systems and methods described herein, for a current patient, the surgeon is able to leverage this large universe of historical patient data in order to determine IOL parameters that would result in optimized surgical outcomes for the current patient. Accordingly, the techniques herein improve the medical field by allowing for better IOL parameters to be selected, thereby leading to improved vision after placement of an IOL, such as during cataract surgery.

Various techniques may be used to train and deploy models that generate IOL parameters for a current patient. One example deployment is illustrated in FIG. 1, however, other deployments are also contemplated. For example, FIG. 1 illustrates a deployment in which models are trained on remote server 120 and deployed to user console 130 used by a surgeon during cataract surgery planning. In another example deployment, the models may be trained and deployed on remote server 120, e.g., accessible through a computing system, an imaging device, and/or a surgical console. In another example deployment, the models may be trained on remote server 120 and deployed to an imaging device 110 used pre-operatively and/or intra-operatively. It should be recognized, however, that various other techniques for training and deploying models that generate IOL parameters for a current patient may be contemplated, and that the deployment illustrated in FIG. 1 is a non-limiting, illustrative, example.

FIG. 1 illustrates an example computing environment 100 in which one or more imaging device(s) 110, a server 120, user console 130, and a historical patient data repository 140 are connected via a network in order to train one or more models for use in generating recommended IOL parameters for a current patient based, at least in part, on the data points associated with measurements of anatomical parameters for the current patient, as provided by the one or more imaging device(s) 110.

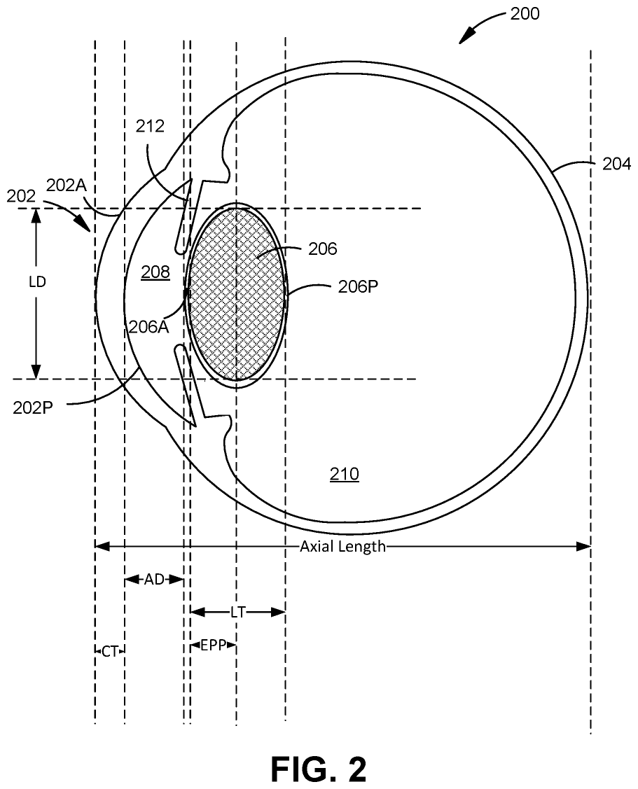

Imaging device(s) 110 are generally representative of various devices that can generate data points associated with one or more measurements of anatomical parameters of a patient's eye. Herein, anatomical parameters of an eye may refer to a set optical parameters. FIG. 2 is a diagram of a model eye 200 illustrating various optical parameters, in accordance with certain aspects described herein. As shown in FIG. 2, the optical parameters may include, the axial length (e.g., the distance from the anterior corneal surface 202A of cornea 202 to the retina 204), a central corneal thickness (CT) measurement of cornea 202, an anterior chamber depth (AD) (e.g., the distance from the posterior cornea 202P apex to the anterior lens surface 206A apex of lens 206) of anterior chamber 208, one or more crystalline lens feature dimensions such as, but not limited to, a lens thickness (LT) of lens 206, a lens diameter (LD) of lens 206 (also referred to as lens equatorial diameter), lens volume of lens 206, lens surface area of lens 206, anterior corneal surface 202A, curvature of the posterior lens surface 206P, white-to-white diameter (WD) (e.g., the distance between the corneal or scleral boundary on each side of the eye), anterior chamber depth (e.g., the distance between anterior vertex of cornea 202 and the anterior vertex of the lens 206), or other crystalline lens feature dimensions), curvature and astigmatism of the anterior corneal surface 202A, the shape of anterior corneal surface 202A, a depth of vitreous humor 210, and other optical parameters associated with the patient's eye, including the patient's crystalline lens feature dimensions, lens capsule geometry, ciliary muscle 212, and ciliary zonules (e.g., such as, but not limited to, ciliary range, ciliary processing moment, ciliary length, and the like).

These optical parameters are examples of anatomical parameters measured by imaging device(s) 110, however, imaging device(s) 110 may measure additional anatomical parameters that may be used in the models.

In some embodiments, data generated by imaging devices 110 for each patient may be stored in repository 140 as patient data, which may include raw measurement values of the patient's anatomical parameters, biometry information derived from measurements, and/or imaging data including two-dimensional cross-sectional images showing the cornea, iris, lens, and retina; three-dimensional images of the eye; two-dimensional topographic maps of the eye; or other types of imaging data. Generally, any number of imaging devices 110 may be included in computing environment 100 and may be used to generate different types of data that may be used as input into one or more models that generate recommended IOL parameters.

Imaging device(s) 110 may include (1) pre-operative measurement and/or imaging devices used in the clinic and/or (2) intra-operative measurement and/or imaging devices. In one example, one of imaging device(s) 110 may be an optical coherence tomography (OCT) device that can generate optical imaging. Various types of OCT devices may be used. As an example, an OCT device may be used to generate a two-dimensional cross-sectional image of the current patient's eye from which measurements of various anatomical parameters may be derived. The two-dimensional cross-sectional image may show the location of the cornea, lens, and retina on a two-dimensional plane (e.g., with the cornea on one side of the two-dimensional cross-section and the back of the retina on the other side of the two-dimensional cross-section). From the two-dimensional cross sectional image of the current patient's eye, the OCT device can derive various measurements.

For example, the OCT device can generate, from the cross-sectional image, an axial length measurement, a central corneal thickness measurement, an anterior chamber depth measurement, a lens thickness measurement, and other relevant measurements. In some aspects, an OCT device may generate one-dimensional data measurements (e.g., from a central point) or may generate three-dimensional measurements from which additional information, such as tissue thickness maps, may be generated. Examples of OCT devices are described in further detail in U.S. Pat. No. 9,618,322 disclosing “Process for Optical Coherence Tomography and Apparatus for Optical Coherence Tomography” and U.S. Pat. App. Pub. No. 2018/0104100 disclosing “Optical Coherence Tomography Cross View Image”, both of which are hereby incorporated by reference in their entirety.

Another one of imaging device(s) 110 may be a keratometer. Generally, a keratometer may reflect a light pattern, such as a ring of illuminated dots, off the current patient's eye and capture the reflected light pattern. The keratometer can perform an image analysis on the reflected pattern (relative to pattern output by the imaging device 110 for reflection from the current patient's eye) to measure or otherwise determine values for various anatomical parameters. These anatomical parameters may include, for example, curvature information and astigmatism information for the front corneal surface of the current patient's eye. The curvature information may, for example, be a general curvature measurement, a maximum curvature and axial information identifying the axis along which the maximum curvature occurs, and a minimum curvature and axial information identifying the axis along which the minimum curvature occurs.

Yet another one of the imaging device(s) 110 may be a topography device that measures the topography of the anterior corneal shape. The topography device may use a reflected light pattern analysis distributed over the corneal region to generate a detailed surface profile map relative to a base profile. For example, as the cornea is typically spherical or nearly spherical, the surface profile map can show deviations from the base profile, where different colors represent an amount of deviation from the base profile at any discrete point along the cornea.

Another example imaging device 110 may be an intra-operative aberrometer. An example of an intra-operative aberrometer is Ora™ with Verifeye™ (Alcon Inc., Switzerland), which is partially described in more detail in commonly owned U.S. Pat. No. 7,883,505 disclosing “Integrated Surgical Microscope and Wavefront Sensor” and U.S. Pat. No. 8,784,443 disclosing “Real-Time Surgical Reference Indicium Apparatus and Methods for Astigmatism Correction”, both of which are hereby incorporated by reference in their entirety. Ora™ can, among other measurements, capture a total ocular refraction measurement that accounts for total ocular astigmatism, including surgically-induced astigmatism and posterior corneal astigmatism, as well as post-myopic PRK/LASIK and long and short eye measurements to provide guidance for adjustments of lens selection and placement for all eye types.

Imaging device(s) 110 may also include a rotating camera (e.g., a Scheimpflug camera), a magnetic resonance imaging (MRI) device, an ophthalmometer, an optical biometer, a three-dimensional stereoscopic digital microscope (such as NGENUITY® 3D Visualization System (Alcon Inc., Switzerland).

Server 120 is generally representative of a single computing device or cluster of computing devices on which training datasets can be generated and used to train one or more models for generating recommended IOL parameters. Server 120 is communicatively coupled to historical patient data repository 140 (hereinafter “repository 140”), which stores records of historical patients. In certain embodiments, repository 140 may be or include a database server for receiving information from server 120, user console 130, and/or imaging devices 110 and storing the information in corresponding patient records in a structured and organized manner.

In certain aspects, each patient record in repository 140 includes information such as the patient's demographic information, data points associated with measurements of anatomical parameters, actual treatment data associated with the patient's cataract surgery, and treatment results data. For example, the demographic information for each patient includes patient age, gender, ethnicity, and the like. The data points associated with pre-operative and/or intra-operative measurements of anatomical parameters may include raw data generated by or measurements derived from or provided by an OCT device, a keratometer, a topography device, etc., as discussed above. As further elaborated below, the actual treatment data includes the actual IOL parameters (e.g., IOL type, IOL power, IOL size) of an IOL used for the patient, as well as any additional relevant information relating to the treatment of the patient. For example, the actual treatment data may indicate the method of performing the cataract surgery for the patient, the tools that were used for the treatment, and other information about the specific procedures performed during the surgery. Each patient record also includes treatment result data, which may include various data points indicative of result parameters, such as the patient's satisfaction with the treatment such as a binary indication of satisfaction or dissatisfaction with the results of the surgery, measured vision levels after treatment, or the like.

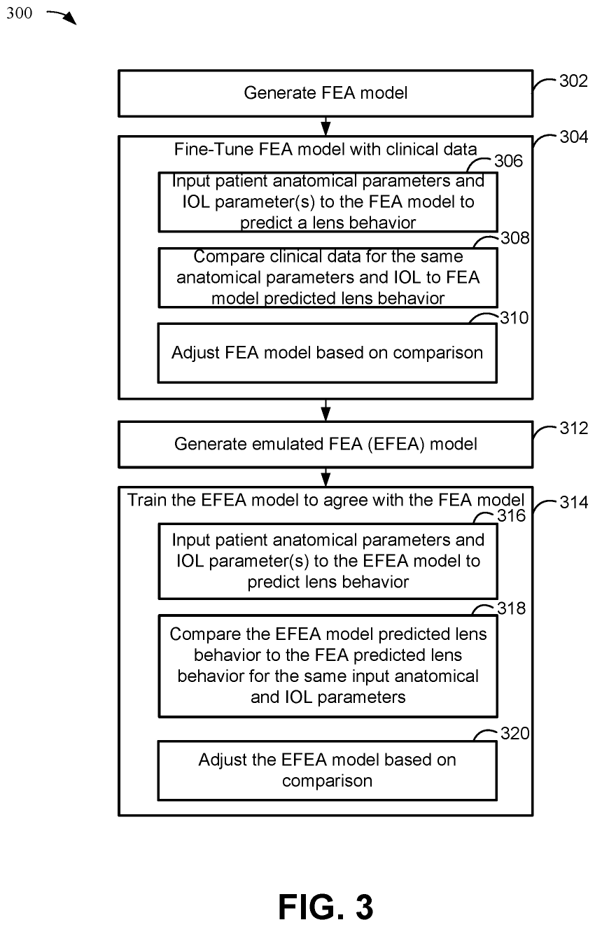

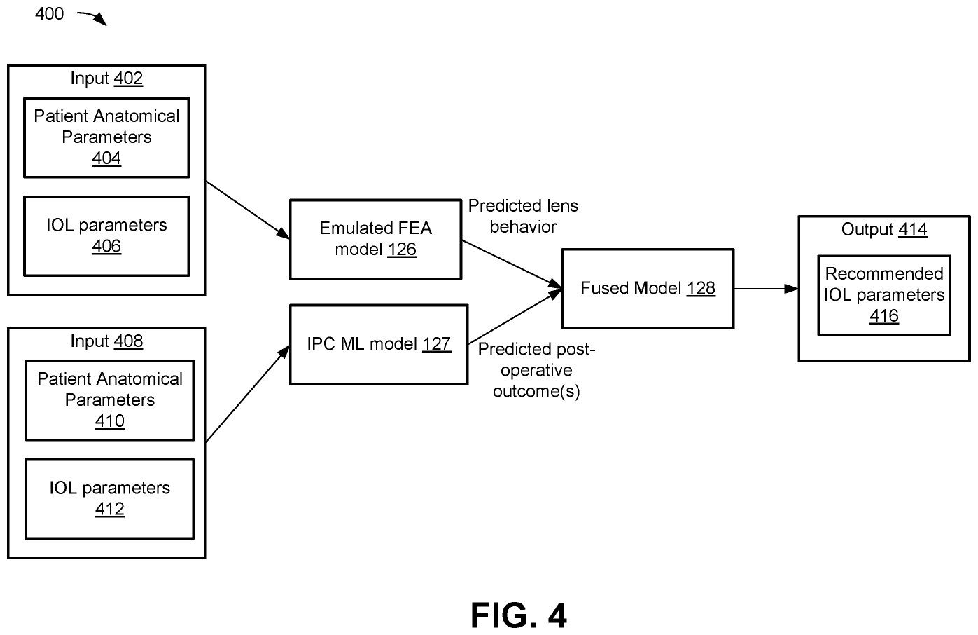

Server 120 uses these records of historical patients to generate datasets for use in training models that can recommend IOL parameters to a surgeon for treating a current patient. More specifically, as illustrated in FIG. 1, server 120 includes one or more training data generator(s) 122 (hereinafter “TDG 122”) and one or more model trainer(s) 124. TDG 122 retrieves data from repository 140 to generate datasets for use by model trainer(s) 124 to train an FEA model 125, an EFEA model 126, an IPC ML model 127, and/or a fused model 128. It should be understand that FEA model 125, EFEA model 126, IPC ML model 127, and fused model 128 may be trained with the same, overlapping, or different datasets. Further, FEA model 125, EFEA model 126, IPC ML model 127, and fused model 128 may be trained by different model trainers 124. Generation and training of FEA model 125 and EFEA model 126 is discussed in more detail below with respect to FIG. 3. Generation and training of IPC ML model 127 and fused model 128 is discussed in more detail below with respect to FIG. 4.

Model trainer(s) 124 include or refer to one or more algorithms that are configured to use training datasets to train FEA model 125, EFEA model 126, IPC ML model 127, and fused model 128. In certain embodiments, a trained model refers to a function, e.g., with weights and parameters, that is used to make IOL-related predictions. IOL-related predictions may include predicted post-operative refractive error, predicted optimal IOL parameters, and IOL power per frame.

Model trainer(s) 124 may use one or more ML algorithms to train one or more of EFEA model 126, IPC ML model 127, and fused model 128. An ML algorithm may generally include a supervised learning algorithm, an unsupervised learning algorithm, and/or a semi-supervised learning algorithm. Unsupervised learning is a type of ML algorithm used to draw inferences from datasets consisting of input data without labeled responses. Supervised learning is a ML task of learning a function that, for example, maps an input to an output based on example input-output pairs. Supervised learning algorithms, generally, include regression algorithms, classification algorithms, decision trees, various types of neural networks, etc.

In some embodiments, model trainer(s) 124 may train deep learning models. These deep learning models may include, for example, convolutional neural networks (CNNs), adversarial learning algorithms, generative networks, or other deep learning algorithms that can learn relationships in data sets that may not be explicitly defined in the data used to train such models. Deep learning models may, for example, map an input to different neurons in one or more layers of the deep learning model (e.g., where the models are generated using neural networks), where each neuron in the model represents new features in an internal representation of an input that are learned over time. These neurons may then be mapped to an output representing recommended IOL parameters, as discussed above.

After the models are trained, model trainer(s) 124 may deploy one or more of the trained models to user console 130 for use in predicting and recommending IOL parameters for a current patient. In some embodiments, EFEA model 126, IPC ML model 127, and fused model 128 are deployed on user console 130, butFEA model 125 is not. For example, as discussed herein, FEA model 125 may be computationally expensive and may require a specialist to generate and run. Instead, as discussed in more detail below with respect to FIG. 3, FEA model 125 may be used to generate the less computationally expensive and simpler to operate EFEA model 126 which is then deployed on user console 130. As described above, FIG. 1 only illustrates one example of where EFEA model 126, IPC ML model 127, and fused model 128 may be deployed. In other examples, EFEA model 126, IPC ML model 127, and fused model 128 may be deployed to or executed at server 120. In yet other examples, model trainer(s) 124 may deploy EFEA model 126, IPC ML model 127, and fused model 128 to one or more of imaging device(s) 110.

User console 130 is generally representative of a computing device or system that is communicatively coupled to server 120, repository 140, and/or imaging device(s) 110. In certain embodiments, user console 130 may include or be associated with a desktop computer, laptop computer, tablet computer, smartphone, or other computing device(s). For example user console 130 may include or be associated with a computing system used at the surgeon's office or clinic. In another example, user console 130 may be a surgical console used by a surgeon in an operating room to perform cataract surgery for a current patient.

In the example of FIG. 1, the trained models are deployed by server 120 to user console 130 for predicting, for a current patient, IOL parameters that would optimize the patient's surgical outcomes. As illustrated, user console 130 includes a treatment data recorder (TDR) 132 and an IOL recommendation generator (IRG) 134. Note that although in FIG. 1, TDR 132 and IRG 134 execute on user console 130, in certain other embodiments, TDR 132 and IRG 134 may execute on one or more other computing systems, such as server 120, an imaging device 110, and/or any computing system that is able to communicate with one or more of server 120, imaging device 110, and/or historical patient data repository 140.

IRG 134 generally refers to a software module or a set of software instructions or algorithms, including the EFEA model 126, IPC ML model 127, and fused model 128, which take a set of inputs about a current patient and generate, as output, recommended IOL parameters. In certain embodiments, IRG 134 is configured to receive the set of inputs from at least one of repository 140, imaging device(s) 110, a user interface of user console 130, and other computing devices that a medical team may use to record information about the current patient. In certain embodiments, IRG 134 outputs the recommended IOL parameters to a display device communicatively coupled with user console 130, prints the recommended IOL parameters, generates and transmits one or more electronic messages, including the recommended IOL parameters, to a destination device (e.g., a connected device, such as a tablet, smartphone, wearable device, etc.), or the like.

The set of inputs described above include data points associated with measurements of the anatomical parameters of the new patient's eye, as provided by imaging device(s) 110. These data points are provided to user console 130 to be used as input to the models and may also be provided to server 120 and/or repository 140.

Prior to or during surgery, user console 130 retrieves the data points associated with pre-operative and/or intra-operative measurements of anatomical parameters (e.g., from the repository 140 or from temporary memory at the user console 130) and other patient information (e.g., the current patient's demographic information, etc.) associated with the current patient to use as inputs into the models stored at user console 130. The data points and other patient information may be provided to IRG 134, which runs the inputs through EFEA model 126, IPC ML model 127, and fused model 128 to generate recommended IOL parameters. User console 130 then outputs the recommendations generated by one or more of the models.

TDR 132 receives or generates treatment data regarding the treatment provided to the current patient. As described above, treatment data may include the actual IOL parameters used for the current patient, as well as any additional relevant information, such as the method of performing the cataract surgery etc. As previously defined, actual IOL parameters refer to the type, power, and size information for the IOL that the surgeon actually implanted in the current patient's eye. In cases where the surgeon does not follow the recommended IOL parameters, the actual IOL parameters would be different from the recommended IOL parameters. In certain such cases, TDR 132 may receive treatment data as user input to a user interface of user console 130. In cases where the surgeon follows the recommended IOL parameters, the actual IOL parameters would be the same as the predicted IOL parameters. In certain such cases, TDR 132 treats the recommended IOL parameters as the actual IOL parameters that are recorded as part of the treatment data. In the embodiments of FIG. 1, TDR 132 transmits the actual IOL parameters to repository 140 and/or server 120. Repository 140 then augments the current patient's record with the actual IOL parameters used for the treatment.

TDR 132 generally allows a user of user console 130 to provide post-surgical information identifying surgical outcomes of the treatment. While TDR 132 is illustrated as executing on user console 130, it should be recognized by one of skill in the art that TDR 132 can execute on a computing device separate from user console 130.

TDG 122 continues to augment the training datasets with information relating to patients for whom the deployed models provided recommended IOL parameters. In certain aspects, TDG 122 augments the dataset(s) every time information about a new (i.e., current) patient becomes available. In certain other aspects, TDG 122 augments the dataset(s) with a batch of new patient records, which may be more resource efficient. TDG 122 may convert a record in repository 140, including the data points associated with the current patient's measurements for one or more anatomical parameters, the actual treatment data, and the treatment result data, into a new sample in a training data set. Model trainer(s) 124 use the new training data set to retrain one or more of the models. More generally, each time a new (i.e., current) patient is treated, information about the new patient may be saved in repository 140 for TDG 122 to supplement the training data set(s), and model trainer(s) 124 use the supplemented training data set(s) to retrain one or more of the models.

### Example Method for Generating an Emulated FEA Model

FIG. 3 is a flow diagram illustrating example operations 300 for training of an emulated finite elements analysis model (e.g., EFEA model 126), in accordance with certain aspects described herein. Operations 300 may be performed by one or more model trainers 124 illustrated in FIG. 1.

As illustrated, operations 300 begin at block 302, by generating a FEA model. In some embodiments, FEA model 125 is generated by a model trainer 124. Generating the FEA model may include generating a mathematical model of interactions of components of the human eye (e.g., model eye 200) with an IOL, such as an accommodating lens. In some embodiments, the FEA model comprises a set of PDEs and/or matrices that can be solved to compute one or more quantities associated with the interactions of the accommodating IOL and the components of the human eye. The variables of the mathematical model include pre-operative anatomical parameters of the eye. For example, the variables may include any of the anatomical parameters discussed above measured by imaging device(s) 110. In particular, the anatomical parameters include, but are not limited to, the parameters discussed above associated with the eye, the crystalline lens feature dimensions, and the lens capsule geometry. Crystalline lens feature dimensions may be used to define a complete pre-operative profile of the lens capsule. Use of additional anatomical parameters may add to the accuracy of the FEA model.

In some embodiments, the FEA model may be solved for one or more sets of anatomical parameters to generate data indicative of how an IOL may behave in a lens capsule, given the IOL's different parameters and characteristics. The FEA model also takes one or more parameters of an IOL as input. For example, the IOL type, size, and/or label power may be input to the FEA model along with each set of anatomical parameters. As used herein, the IOL label power may be the pre-operative power of the IOL.

For an input set of patient pre-operative anatomical parameters and IOL parameters, the FEA model predicts the post-operative behavior of each input IOL after implantation in a patient's capsular bag. In some embodiments, the FEA model predicts a post-operative rest state and effective lens position (e.g., the post-operative position of the IOL relative to the cornea and retina) of the IOL and, thereby, the refractive outcome of the IOL. For example, based on the post-operative effective lens position, the power needed to ensure that light is properly focused on the retina can be determined. The post-operative rest state of the IOL may determine pressure applied to the IOL (e.g., by the haptics as the haptics bend in response to the pressure) and the pressure applied to the IOL changes the shape and size of the IOL, which in turn, affects the IOL power. Accordingly, by the FEA model predicting the post-operative rest state and effective lens position, the FEA model can predict the sizing factor and post-operative refractive outcome of the IOL. As discussed in more detail with respect to FIG. 3, the FEA model may output a predicted IOL power per frame. As used herein, a frame refers to a measurement instance in time. Accordingly, the FEA model may output predicted IOL power at different points in time, thereby modeling how the accommodating IOL interacts with the patient's capsular bag after implantation in the patient's eye.

As illustrated, operations 300 may continue, at block 304, by fine-tuning the FEA model with clinical data. As illustrated, fine-tuning the FEA model with clinical data, at block 304, includes inputting historical patient anatomical parameters and one or more IOL parameters to the FEA model to predict IOL behavior based on the input at block 306. In some embodiments, the historical patient anatomical parameters input to the FEA model includes values for each of the anatomical parameters used to build the FEA model. In some embodiments, the patient anatomical parameters input to the FEA model includes values for a subset of the anatomical parameters used to build the FEA model. In one illustrative embodiment, the historical patient anatomical parameters include the historical patient's pre-operative crystalline lens feature dimensions and the IOL parameters are one or more IOL label powers. Based on the inputs, the FEA model outputs a predicted IOL behavior. In one illustrative embodiment, the predicted IOL behavior includes IOL power per frame.

As illustrated, fine-tuning the FEA model with clinical data, at block 304, includes comparing clinical data for the same anatomical parameters to the FEA model's predicted IOL behavior at block 308. For example, where the FEA model 125 outputs predicted IOL power per frame for an input set of anatomical parameters and IOL parameters, the predicted IOL power per frame is compared to clinical results (e.g., treatment results in repository 140), which may include actual IOL power per frame for a patient with the same set of anatomic parameters and the same IOL parameters. At block 310, the FEA model is adjusted based on the comparison. In some embodiments, the FEA model is adjusted by varying one or more parameters of the FEA model. Parameters of the FEA model may include, but are not limited to, zonular tension, zonular dynamics, capsule elasticity, friction, and/or other parameters of the FEA model.

FEA model 125 may be fine-tuned across many sets of anatomical parameters and IOL parameters to find the FEA model that accurately predicts the IOL behavior. As used herein, the FEA model that accurately predicts the IOL behavior may predict the IOL behavior at a specified threshold level of success. In some embodiments, FEA model 125 is fine-tuned across a complete range, or a larger range, of anatomical values expected to be seen in a clinic.

Referring back to FIG. 3, after fine-tuning the FEA model, operations 300 continue, at block 312, by generating an emulated FEA model (e.g., EFEA model 126). The EFEA model is generated to match (e.g., or closely match within a specified threshold) the FEA model. In some embodiments, the EFEA model is a machine-learning model.

Operations 300 continue at block 314, by training the EFEA model to replicate the output of the FEA model. In some embodiments, EFEA model 126 is trained by a model trainer 124 illustrated in FIG. 1. In some embodiments, a model trainer 124 refers to an AI/ML learning algorithm or a combination of AI/ML learning algorithms for training AI/ML models. Examples of AI/ML learning algorithms include various types of optimization algorithms such as gradient descent, stochastic gradient descent, non-linear conjugate gradient, etc., The EFEA model 126 may be Gaussian process regression (GPR) model, linear regression model, autoregressive integrated moving average (ARIMA) model, a neural network, or the like. For input values where FEA model 125 was run, EFEA model 126 is expected to return the same output values with zero uncertainty. For input values which FEA model 125 was not run, EFEA model 126 predicts what FEA model 125 would output along with an estimated uncertainty.

As illustrated in FIG. 3, training the EFEA model to replicate the output of the FEA model at block 314 includes inputting historical patient anatomical parameters and one or more IOL parameters to the EFEA model to predict IOL behavior based on the input at block 316. In order to train EFEA model 126 to emulate the FEA model 125, the input parameters are input parameters that were run by the FEA model 125.

Training the EFEA model to agree with the FEA model at block 314 further includes comparing the EFEA model's predicted IOL behavior to the FEA models' predicted IOL behavior with the same input, at block 316. As discussed above, in some embodiments, the predicted IOL behavior by FEA model 125 and EFEA model 126 is IOL power per frame. Accordingly, in some embodiments, EFEA model 126 may be trained to reproduce the FEA predicated IOL power per frame curves. Training the EFEA model to agree with the FEA model at block 314 further includes adjusting the EFEA model (e.g., adjust the model's weights) based on the comparison at block 318.

TDG 122 may generate a training data set (or multiple training data sets) used to train EFEA model 126. The training data set for EFEA model 126 may include mapping the demographic information and/or data points associated with measurements of anatomical parameters for each historical patient of a number of historical patients to corresponding predictions of IOL behavior by the FEA model 125. A model trainer 124 trains EFEA model 126 based on the training dataset(s). For example, the model trainer 124 trains EFEA model 126 to generate a predicted IOL behavior (e.g., IOL power per frame) for a given input, including the anatomical parameters and IOL parameter(s). EFEA model 126 then provides a predicted IOL behavior, based, at least in part on the input. Repository 140 may include records of the predictions by FEA model 125 of IOL behavior for the same set of inputs. The predicted IOL behavior provided by EFEA model 126 can be compared to the record of the outputs provided by FEA model 125 for the same set of inputs for minimizing the loss between the predictions provided by EFEA model 126 and the outputs provided by FEA model 125. Once EFEA model 126 is trained to a point where it returns predictions that are the same or almost the same (e.g., within an acceptable margin of error) as the outputs provided by FEA model 125 for the same set of inputs, then EFEA model 126 may be capable of extrapolating and providing predicted IOL behavior even for inputs that were not run through FEA model 125. As a result, training EFEA model 126 according to the embodiments described herein addresses a technical deficiency in using FEA models for predicting IOL behavior in a lens capsule by significantly reducing the amount of resources (e.g., compute resources) that would otherwise have to be utilized for predicting IOL behavior over a large range of inputs with an FEA model.

As discussed above, FEA model 125 may be computationally expensive and require a specialist to generate, set up, and run. EFEA model 126, however, can return predictions in a fraction of a second with minimal software and hardware requirements. Thus, according to embodiments of the present disclosure, EFEA model 126, and not FEA model 125, is used as part of IOL recommendation generator 134 to generate recommended IOL parameters. Accordingly, after training EFEA model 126, EFEA model 126 may be deployed to one or more server computers, a user console, an imaging device in which a computing device is integrated, or the like. As illustrated for example in FIG. 1, EFEA model 126 may be deployed to user console 130.

### Example Method for Training a Fused Model

According to embodiments of the present disclosure, an EFEA (e.g., EFEA model 126) may be used in addition to an IPC ML based model (e.g., IPC ML model 127) to train a fused model to generate predicted IOL behavior and/or recommended IOL parameters.

FIG. 4 illustrates use of the EFEA model 126 and IPC ML model 127 to train fused model 128, in accordance with certain aspects described herein. Training of EFEA model 126 is discussed above with respect to FIG. 3. To train the fused model 128, EFEA model 126 may be run and the output of EFEA model 126 may be used as input to the fused model 128. EFEA model 126 uses input 402 to generate predicted IOL behavior. Input 402 to the EFEA model 126 includes patient anatomical parameters 404 and IOL parameters 406. As discussed above, patient anatomical parameters 404, which may be input to EFEA model 126 may include all or a subset of the anatomical parameter used to generate FEA model 125. IOL parameters 406 may include IOL type, IOL size, and/or IOL label power. Based on the inputs, EFEA model 126 outputs predicted IOL behavior. As discussed above, the predicted IOL behavior may include a predicted post-operative refractive error, a predicted post-operative effective lens position, and/or a predicted IOL power frame (which may include predicted pre-operative, intra-operative, and post-operative IOL power). In an illustrative embodiment, patient anatomical parameters 604 includes crystalline lens feature dimensions; IOL parameters 406 includes at least the IOL label power for one or more IOLs; and the predicted IOL behavior includes at least predicted IOL power per frame for each of the one or more IOLs.

The output from an IPC is also used as input to fused model 128, in addition to the output from EFEA model 126. As discussed above, the IPC may be a standard IPC (e.g., a Bartlett IPC, a Haigis IPC, a Hill-RBF IPC, an SRK IPC, or the like). In some embodiments, IPC ML model 127 is used.

TDG 122 may generate a training data set (or multiple training data sets) used to train IPC ML model 127. The training data set for IPC ML model 127 may include mapping the demographic information and/or data points associated with measurements of anatomical parameters for each historical patient of a number of historical patients to corresponding clinical results of the post-operative outcomes. In some embodiments, the training data sets for IPC ML model 127 involve the same pre-operative anatomical parameters as used for the EFEA model 126. In some embodiments, the training data set for IPC ML model 127 uses a subset of the anatomical parameters as the EFEA model 126. That is, in some embodiments, FEA model 125 and EFEA model 126 may use all of the anatomical parameters of the IPC ML model 127 as well as additional modeled anatomical parameters.

A model trainer 124 trains IPC ML model 127 based on the training dataset(s). For example, the model trainer 124 trains IPC ML model 127 to generate a predicted post-operative outcome for a given input 408, including the patient anatomical parameters 410 and IOL parameter(s) 412. In some embodiments, patient anatomic parameters 410 of input 408 to IPC ML model 127 are the same patient anatomical parameters 404. In some embodiments, patient anatomic parameters 410 of input 408 to IPC ML model 127 are a subset of patient anatomical parameters 404. IPC ML model 127 outputs a predicted post-operative outcome, based, at least in part on the input. In some embodiments, the predicted post-operative outcome is a predicted post-operative refractive error, a predicted post-operative effective lens position, and/or a predicted IOL power. In some embodiments, IPC ML model 127 outputs a predicted IOL per frame per IOL based, at least in part on the input.

To train and refine the IPC ML model 127, the predicted post-operative outcomes by IPC ML model 127 can be compared to historical clinical data records in repository 140 of treatment results for the same set of inputs to determine whether the predictions by IPC ML model 127 provided a satisfactory result. Based on the comparison, model trainer 124 may adjust the IPC ML model 127 to improve the model. IPC ML model 127 may be trained over a large set of anatomical values and IOL parameters.

After being trained, IPC ML model 127 may be deployed to one or more server computers, a user console, a imaging device in which a computing device is integrated, or the like. As illustrated for example in FIG. 1, IPC ML model 127 may be deployed to user console 130.

Fused model 128 may be another ML-based model trained with input from both the EFEA model 126 and the IPC ML model 127 to generate recommended IOL parameters. In some embodiments, fused model 128 may be trained to weigh input from EFEA model 126 differently than input from IPC ML model 127. In some embodiments, the fused model 128 may weight inputs from EFEA model 126 and/or IPC ML model 127 differently for different data points.

Output 414 from fused model 128 includes at least one or more recommended IOL parameters 416. As discussed herein, the one or more recommended IOL parameters 416 may include recommended IOL type, IOL power, and/or IOL size.

In some other embodiments, output 414 from fused model 128 may include one or more predicted post-operative outcome(s), such as a predicted post-operative refractive error for each of one or more IOLs.

Prior to deployment, fused model 128 may go through a training period, involving a model trainer 124 adjusting the weights of fused model 128 to reduce the loss calculated between output 414, which may indicate a predicted post-operative refractive error. Fused model 128 may be trained to recommend IOL parameters that result in a lowest predicted post-operative refractive error. The treatment results data, which may indicate the actual post-operative refractive errors measured for the patient post-operatively, across a large set of inputs. Treatment results data, which may be provided as part of each historical patient record, may generally indicate the actual IOL used for the patient, post-operative measurement results (e.g., the actual post-operative refractive errors measured for the patient post-operatively), and/or post-operative client satisfaction score.

Once trained, fused model 128 may be deployed to one or more server computers, a user console, an imaging device in which a computing device is integrated, or the like. As illustrated for example in FIG. 1, fused model 128 may be deployed to user console 130. After deployment, fused model 128 may continue to be trained similarly using the treatment results data recorded for current/new patients by TDR 132 and added to repository 140.

### Example Method for Performing Ophthalmic Surgery Based on Recommendations Generated Using a Fused Model

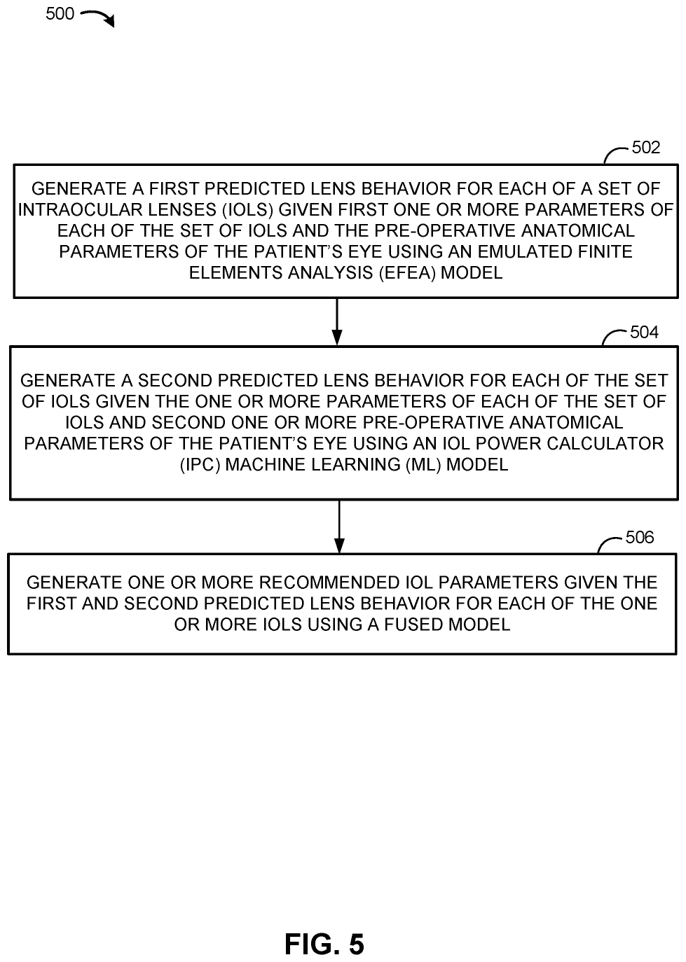

FIG. 5 illustrates example operations 500 that may be performed by a computing device to generate and output recommendations, including IOL parameters, for a patient's cataract surgery based on the patient's anatomical parameters, in accordance with certain aspects described herein. Operations 500 may be performed by a clinical device, such as user console 130.

Operations 500 may begin, at block 502, by generating a first predicted lens behavior (e.g., IOL power per frame) for each of a set of IOLs given first pre-operative anatomical parameters (e.g., patient anatomical parameters 404) of a patient's eye and one or more parameters (e.g., IOL parameters 406) of each of one or more IOLs using an EFEA model (e.g., EFEA model 126). In an illustrative embodiment, for a current patient in a clinic, a clinician inputs the patient's pre-operative anatomical parameters including the patient's crystalline lens feature dimensions into user console 130. In some embodiments, user console 130 itself measures the patient's anatomical parameters. In some embodiments, a imaging device 110 measures the patient's anatomical parameters and the imaging device 110 provides the measured anatomical parameters to user console 130 or displays the measured anatomical parameters to the clinician who manually enters the anatomical parameters to user console 130. In some embodiments, the patient's anatomical parameters were previously measured and stored, and user console 130 retrieves the stored anatomical parameters or the clinician retrieves the stored anatomical parameters and manually enters the anatomical parameters to user console 130. In some embodiments, the one or more IOLs comprises a set of accommodating IOLs, such as a set of IOLs in inventory or available to the clinician. The set of IOLs may be IOLs covering a range of IOL powers at a particular IOL power step-size.

The EFEA model outputs a first predicted lens behavior for each of the one or more IOLs. In the illustrative example, based on the input pre-operative anatomical parameters of the patient, EFEA model 126 outputs predicted IOL power for a plurality of frames for each of the input IOLs and the pre-operative anatomical parameters. The plurality of frames may include at least frames starting from implantation of the IOL in the patient's eye until the IOL reaches a rest state.

Operations 500 may continue, at block 504, by generating a second predicted lens behavior (e.g., post-operative refractive outcome) for each of the set of IOLs given second one or more pre-operative anatomical parameters (e.g., patient anatomical parameters 410) of the patient's eye and one or more parameters (e.g., IOL parameters 412) of each of one or more IOLs using an IPC ML model (e.g., IPC ML model 127). In some embodiments, the anatomical parameters input to IPC ML model 127 are the same, or a subset of, the anatomical parameters input to EFEA model 126 at block 502. The IPC ML model outputs a second predicted post-operative outcome for each of the one or more IOLs. In some embodiments, based on the input pre-operative anatomical parameters and IOL parameters, IPC ML model 127 outputs predicted post-operative refractive error for each of the one or more IOLs, a predicted IOL power for each of the one or more IOLs, a predicted IOL power per frame for each of the one or more IOLs, or a combination thereof.

Operations 500 may continue, at block 506, by generating one or more recommended IOL parameters (e.g., recommended IOL parameters 416) given the first and second predicted lens behavior for each of the one or more IOLs using a fused model (e.g., fused model 128). In some embodiments, fused model 128 outputs a recommended IOL type, power, and/or size based on the inputs from EFEA model 126 and IPC ML model 127.

### Example System for Performing Ophthalmic Surgery Based on Recommendations Generated Using a Fused Model

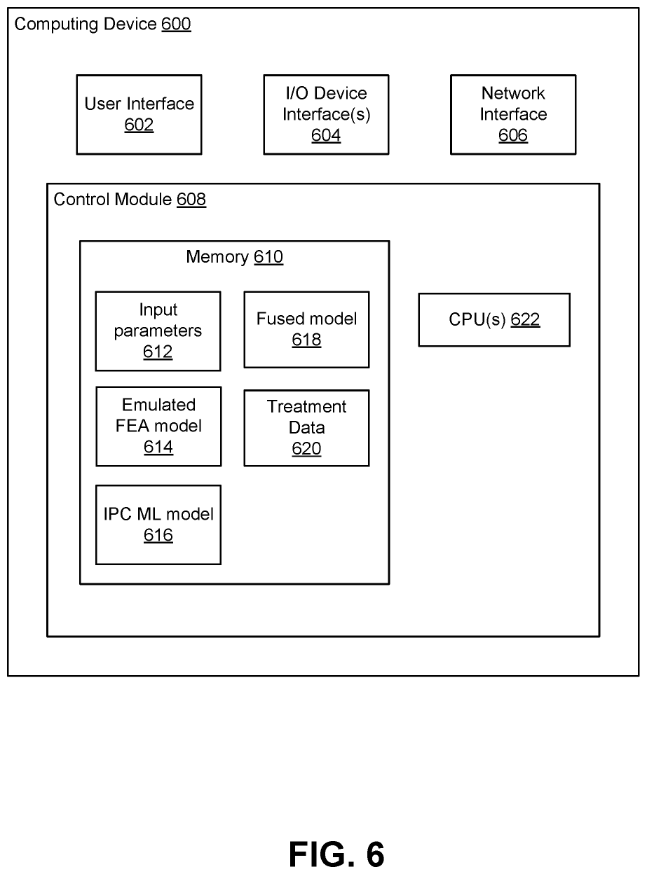

FIG. 6 illustrates an example computing device 600 that uses an EFEA model (e.g., such as EFEA model 126) to aid in performing ophthalmic procedures, such as cataract surgeries, in accordance with certain aspects described herein. For example, computing device 600 may be user console 130 illustrated in FIG. 1.

As shown, computing device 600 includes a user interface 602, one or more input/output (I/O) interfaces 604, a network interface 606, and a control module 608.

User interface 602 may be a graphical user interface (GUI) through which a user interacts with computing device 600. I/O interfaces 604 may allow for the connection of various I/O devices (e.g., keyboards, displays, mouse devices, pen input, etc.) to the computing device 600. Network interface 606 may connect computing device 600 is connected to a network (which may be a local network, an intranet, the internet, or any other group of computing devices communicatively connected to each other).

Control module 608 includes memory 610, central processing unit(s) 622, and storage 624. CPU(s) 622 may retrieve and execute programming instructions stored in the memory 610. CPU(s) 622 are representative of a single CPU, multiple CPUs, a single CPU having multiple processing cores, and the like.

Memory 610 is representative of a volatile memory, such as a random access memory, and/or a nonvolatile memory, such as nonvolatile random access memory, phase change random access memory, or the like. Memory 610 may include input parameters 612, EFEA model 614, ML IPC model 616, fused model 618, and treatment data 620.

Computing device 600 may receive input from user interface 602 and/or I/O device interface(s) 604 and store input parameters 612 in memory 610. CPU(s) 622 may retrieve the input parameters 612 and execute EFEA model 614 and ML IPC model 616 with the input parameters 612 to generate first and second predicted post-operative outcomes. CPU(s) 622 may execute fused model 618 with the first and second predicted post-operative outcomes to generate recommended IOL parameters or use in a cataract surgery for a patient. Computing device 600 may receive treatment data from user interface 602 and/or I/O device interface(s) 604 and store the treatment data 620 in memory 610.

### ADDITIONAL CONSIDERATIONS

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method of determining one or more intraocular lens (IOL) parameters for an IOL to be used in a cataract surgery procedure, comprising:
generating, using one or more ophthalmic imaging devices, a plurality of data points associated with measurements of a plurality of anatomical parameters for an eye to be treated;
generating, using a machine learning model trained to emulate a finite elements analysis (FEA) model, first predicted lens behavior based, at least in part, on the plurality of data points associated with the measurements of the plurality of anatomical parameters and one or more IOL parameters for each of one or more IOLs;
generating, using an IOL power calculator machine learning model, second predicted lens behavior based, at least in part, on at least a subset of the plurality of data points associated with the measurements of the plurality of anatomical parameters for the eye to be treated and the one or more IOL parameters for each of one or more IOLs; and
generating, using a fused machine learning model, recommendations including one or more IOL parameters for the IOL to be used in the cataract surgery based, at least in part, on the first and the second predicted lens behavior.

2. The method of claim 1, wherein the plurality of anatomical parameters comprises one or more crystalline lens feature dimensions of the eye.

3. The method of claim 1, wherein the one or more IOL parameters comprises at least one of: an IOL type, an IOL size, or an IOL power.

4. The method of claim 1, further comprising:
generating an FEA model using a finite element method (FEM) based on a set of data points associated with anatomical parameters of at least one historical patient and least one set of one or more IOL parameters;
using the FEA model to generate predicted lens behavior based, at least in part, on the set of data points associated with the anatomical parameters of the at least one historical patient and the at least one set of one or more IOL parameters; and
adjusting the FEA model based on a comparison of the predicted lens behavior to observed lens behavior for an IOL with the at least one set of IOL parameters implanted in the historical patient's eye with the set of data points associated with the anatomical parameters.

5. The method of claim 1, further comprising training the machine learning model to emulate the FEA model by:
using the machine learning model to generate predicted lens behavior based, at least in part, on a set of data points associated with anatomical parameters of at least one historical patient and at least one set of one or more IOL parameters; and
adjusting weights associated with the machine learning model based on a comparison of the predicted lens behavior output by the machine learning model to predicted lens behavior output by the FEA model for the set of data points associated with the anatomical parameters and the at least one set of one or more IOL parameters.

6. The method of claim 5, wherein the predicted lens behavior comprises IOL power per frame.

7. The method of claim 1, further comprising training the IOL power calculator machine learning model by:
using the IOL power calculator machine learning model to generate predicted lens behavior based, at least in part, on a set of data points associated with anatomical parameters of at least one historical patient and at least one set of one or more IOL parameters; and
adjusting the IOL power calculator machine learning model based on a comparison of the predicted lens behavior to observed lens behavior for an IOL with the at least one set of one or more IOL parameters implanted in the historical patient's eye with the set of data points.

8. The method of claim 7, wherein the predicted lens behavior comprises predicted post-operative refractive outcome.

9. The method of claim 1, further comprising training the fused model by:
using the fused model to generate one or more recommended IOL parameters for a historical patient based, at least in part, on a third predicted lens behavior by the machined learning model trained to emulate the FEA model and a fourth predicted lens behavior by the IOL power calculator machine learning model; and
adjusting the fused model based on a comparison of the one or more recommended IOL parameters to treatment result data for an IOL with the recommended one or more IOL parameters implanted in the historical patients' eye.

10. A system for determining one or more intraocular lens (IOL) parameters for an IOL to be used in a cataract surgery procedure, comprising:
one or more ophthalmic imaging devices configured to generate a plurality of data points associated with measurements of a plurality of anatomical parameters for an eye to be treated;
a memory storing a machine learning model trained to emulate a finite elements analysis (FEA) model, an IOL power calculator machine learning model, and a fused machine learning model; and
at least one processor coupled with the memory, the at least one processor configured to:
generate, using the machine learning model trained to emulate the FEA model, first predicted lens behavior based, at least in part, on the plurality of data points associated with the measurements of the plurality of anatomical parameters and one or more IOL parameters for each of one or more IOLs;
generate, using the IOL power calculator machine learning model, second predicted lens behavior based, at least in part, on at least a subset of the plurality of data points associated with the measurements of the plurality of anatomical parameters for the eye to be treated and the one or more IOL parameters for each of one or more IOLs; and
generate, using the fused machine learning model, recommendations including one or more IOL parameters for the IOL to be used in the cataract surgery based, at least in part, on the first and the second predicted lens behavior.

11. The system of claim 10, wherein the plurality of anatomical parameters comprises one or more crystalline lens feature dimensions of the eye.

12. The system of claim 11, wherein the one or more IOL parameters comprise at least one of: an IOL type, an IOL size, or an IOL power.

13. The system of claim 11, wherein the at least one processor is further configured to:
obtain an FEA model generated using a finite element method (FEM) based on a set of data points associated with anatomical parameters of at least one historical patient and least one set of one or more IOL parameters;
use the FEA model to generate predicted lens behavior based, at least in part, on the set of data points associated with the anatomical parameters of the at least one historical patient and the at least one set of one or more IOL parameters; and
adjust the FEA model based on a comparison of the predicted lens behavior to observed lens behavior for an IOL with the at least one set of IOL parameters implanted in the historical patient's eye with the set of data points associated with the anatomical parameters.

14. The system of claim 11, wherein the at least one processor is further configured to train the machine learning model to emulate the FEA model by:
using the machine learning model to generate predicted lens behavior based, at least in part, on a set of data points associated with anatomical parameters of at least one historical patient and at least one set of one or more IOL parameters; and
adjusting weights associated with the machine learning model based on a comparison of the predicted lens behavior output by the machine learning model to predicted lens behavior output by the FEA model for the set of data points associated with the anatomical parameters and the at least one set of one or more IOL parameters.

15. The system of claim 14, wherein the predicted lens behavior comprises IOL power per frame.

16. The system of claim 11, wherein the at least one processor is further configured to train the IOL power calculator machine learning model by:
using the IOL power calculator machine learning model to generate predicted lens behavior based, at least in part, on a set of data points associated with anatomical parameters of at least one historical patient and at least one set of one or more IOL parameters; and
adjusting the IOL power calculator machine learning model based on a comparison of the predicted lens behavior to observed lens behavior for an IOL with the at least one set of one or more IOL parameters implanted in the historical patient's eye with the set of data points.

17. The system of claim 16, wherein the predicted lens behavior comprises predicted post-operative refractive outcome.

18. The system of claim 11, wherein the at least one processor is further configured to train the fused model by:
using the fused model to generate one or more recommended IOL parameters for a historical patient based, at least in part, on a third predicted lens behavior by the machined learning model trained to emulate the FEA model and a fourth predicted lens behavior by the IOL power calculator machine learning model; and
adjusting the fused model based on a comparison of the one or more recommended IOL parameters to treatment result data for an IOL with the recommended one or more IOL parameters implanted in the historical patients' eye.
